SEQUENCE LISTING (7 sequences)

SEQ ID NO: 1 - DNA, 53 bp, Artificial Sequence
  a plasmid insert sequence shown 5′ to 3′
acccgtaaggcaagccanggnnnnnnnnnnccnatcgaccaagtattgcaata

SEQ ID NO: 2 - DNA, 53 bp, Artificial Sequence
  a plasmid insert sequence shown 3′ to 5′
tgggcattccgttcggtnccnnnnnnnnnnggntagctggttcataacgttat

SEQ ID NO: 3 - DNA, 14 bp, Artificial Sequence
  a plasmid insert sequence shown 5′ to 3′
acccgtaaggcaag

SEQ ID NO: 4 - DNA, 17 bp, Artificial Sequence
  a plasmid insert sequence shown 5′ to 3′
gaccaagtattgcaata

SEQ ID NO: 5 - DNA, 14 bp, Artificial Sequence
  a plasmid insert sequence shown 3′ to 5′
tgggcattccgttc

SEQ ID NO: 6 - DNA, 17 bp, Artificial Sequence
  a plasmid insert sequence shown 3′ to 5′
ctggttcataacgttat

SEQ ID NO: 7 - DNA, 11 bp, Artificial Sequence
  a plasmid insert sequence shown 5′ to 3′
gaccaagtatt

(19) United States
(12) Patent Application Publication
(10) Pub. No.: US 2016/0053272 A1
(43) Pub. Date: Feb. 25, 2016

(54) Methods Of Modifying A Sequence Using CRISPR

(71) Applicant: Whitehead Institute for Biomedical Research, Cambridge, MA (US)

(72) Inventors: Omri Wurtzel, Somerville, MA (US); Samuel LoCascio, Boston, MA (US); Peter Reddien, Cambridge, MA (US)

(21) Appl. No.: 14/801,133

(22) Filed: Jul. 16, 2015

Related U.S. Application Data
(60) Provisional application No. 62/026,415, filed on Jul. 18, 2014.

(51) Int. Cl. C12N 15/66

(52) U.S. Cl. CPC C12N 15/66

(57) ABSTRACT

Methods of modifying one or more target nucleic acid sequences using the Clustered Regularly Interspaced Short Palindromic Repeats (CRISPR) and CRISPR associated (Cas) proteins (CRISPR/Cas) system are disclosed. Methods of introducing one or more exogenous nucleic acid sequences into one or more circular nucleic acid sequences using the CRISPR/Cas system are also disclosed.

### RELATED APPLICATION

This Application claims the benefit of U.S. Provisional Application No. 62/026,415, filed on Jul. 18, 2014. The entire teachings of the above application are incorporated herein by reference.

### BACKGROUND OF THE INVENTION

Gibson cloning is a method for assembling two or more DNA fragments with overlapping sequences in a single reaction. Since its publication (Gibson, et. al., Nat. Methods, 2009), it has become recognized for its robust performance in complex and simple cloning scenarios, capable of assembling multiple fragments together without the need for restriction enzyme/ligation or recombinase-based strategies. However, a prerequisite for Gibson assembly cloning is for all substrates to be linear. This requirement prohibits the use of this powerful method in many common scenarios where unique restriction sites cannot be found in the target sequence. For example, modification (e.g., removal, change, or insertion) of a nucleic acid sequence (e.g., a gene, a gene fragment, a tag, a promoter, etc.) in a circular DNA (e.g., plasmid) may be difficult due to a lack of one or more unique restriction sites. In these scenarios it may be difficult to find unique restriction sites that overlap the sequence desired to be modified. Complicated cloning strategies are needed in those instances.

Thus, a need exists for improved methods of modifying (e.g., cloning) nucleic acid sequences e.g., where unique restriction sites are not found.

### SUMMARY OF THE INVENTION

Described herein is the use of the Clustered Regularly Interspaced Short Palindromic Repeats (CRISPR) and CRISPR associated (Cas) proteins (CRISPR/Cas) system to drive precise nucleic acid modification to achieve highly efficient targeting of one or more nucleic acid sequences, including nucleic acid sequences found in plasmids or other circular strands of DNA and RNA.

Accordingly, in one aspect, the invention is directed to a method of modifying one or more target nucleic acid sequences. The method comprises contacting the one or more target nucleic acid sequences with (i) one or more ribonucleic acid (RNA) sequences wherein each RNA sequence comprises a portion that is complementary to all or a portion of one or more of the target nucleic acid sequences, (ii) a (one or more) CRISPR associated (Cas) protein having nuclease activity, (iii) one or more exogenous nucleic acid sequences wherein at least one exogenous nucleic acid sequence comprises a 5′ adapter sequence that hybridizes to a 5′ flanking sequence of the target nucleic acid sequence and at least one exogenous nucleic acid sequence comprises a 3′ adapter sequence that hybridizes to a 3′ flanking sequence of the target nucleic acid sequence, and (iv) a nucleic acid sequence that interacts with Cas protein, thereby producing a combination. The combination is maintained under conditions in which the one or more RNA sequences hybridize to all or the portion of the one or more target nucleic acid sequences to which each RNA sequence forms a complement, thereby forming one or more base paired structures, and the one or more base paired structures and the nucleic acid sequence that interacts with Cas protein directs the Cas protein to cleave the one or more target nucleic acid sequences, thereby modifying the one or more target nucleic acid sequences.

In some aspects, the invention is directed to a method of introducing one or more exogenous nucleic acid sequences into one or more circular nucleic acid sequences. The method comprises contacting the one or more circular nucleic acid sequences with (i) one or more ribonucleic acid (RNA) sequences wherein each RNA sequence comprises a portion that is complementary to all or a portion of one or more target sequences within the one or more circular nucleic acid sequences, (ii) a (one or more) CRISPR associated (Cas) protein having nuclease activity, (iii) one or more exogenous nucleic acid sequences wherein at least one exogenous nucleic acid sequence comprises a 5′ adapter sequence that hybridizes to a 5′ flanking sequence of the target nucleic acid sequence and at least one exogenous nucleic acid sequence comprises a 3′ adapter sequence that hybridizes to a 3′ flanking sequence of the target nucleic acid sequence; wherein at least one exogenous nucleic acid sequence comprises one or more additional nucleotides, and (iv) a nucleic acid sequence that interacts with Cas protein binding site, thereby producing a combination. The combination is maintained under conditions in which the one or more RNA sequences hybridize to all or the portion of the one or more target nucleic acid sequences to which each RNA sequence forms a complement thereby forming one or more base paired structures, and the one or more base paired structures and the nucleic acid sequence that interacts with Cas protein direct the Cas protein to cleaves the target nucleic acid sequence, thereby introducing the one or more exogenous nucleic acid sequence into the one or more circular nucleic acid sequences.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a schematic of a typical Gibson cloning reaction requiring two steps. Reaction 1 shows a circular nucleic acid, with a highlighted sequence in red to be removed. Restrictions enzymes cut the flanking positions (represented by the gray box and solid gray box), thereby linearizing the circular nucleic acid (e.g., plasmid). The nucleic acid products from the restriction enzyme digest are then separated using gel electrophoresis. In reaction 2, the linearized destination vector is combined with an exonuclease, polymerase, and ligase to introduce an exogenous nucleic acid sequence (e.g., partA) into the plasmid.

FIG. 2 is a schematic of the use of a CRISPR/Cas system to remove a target nucleic acid sequence (highlighted red line) and introduce an exogenous nucleic acid sequence (partA).

FIG. 3 is a schematic of modification of one or more fragments of an exemplary plasmid map. The existing clone (left) differs from the desired clone (right) by replacing exon 2 (green arrow in existing clone) with new exon (red arrow in desired clone) and a resistance cassette (KanR in existing clone and CarbR in desired clone). Gibson cloning requires linearization of the plasmid on sites overlapping both exon 2 and the KanR cassette or generation of suitable plasmid fragments by PCR.

FIG. 4 shows use of the methods of the present invention to linearize the plasmid of FIG. 3 with multiple guide RNAs (gRNA; depicted as red arrows) and Cas9. In this example, two nucleic acid fragments are excised during this reaction creating two linearized products.

FIG. 5 shows the cloning of replacement fragments into the clone of FIGS. 3 and 4 linearized by CRISPR. The replacement fragments (red arrows) are flanked by sequences (e.g., plasmid specific adapters) that match their insertion site. The plasmid specific adapters will anneal to the linear plasmid and prime the Gibson assembly reaction.

FIG. 6 shows an exemplary double stranded (ds) DNA sequence on a plasmid. A target sequence of about 20 base pairs and two cut sites adjacent to PAM sequences are shown. A toxic sequence to be replaced is also shown. Below the plasmid sequence is the exogenous fragment to be used for replacement. This sequence is flanked by sequences that are complementary to the resulting linearized plasmid ends. As shown in red, this sequence is part of the target sequence on the plasmid, excluding the PAM and a few bases.

FIG. 7 shows the resulting linearized plasmid after removal of the target nucleic acid sequence within the plasmid using a Cas protein, such as Cas9. Cas9 generates blunt ends, producing a linear plasmid. The fragment (shown below the linearized plasmid) being used for cloning is not affected by Cas9, since it does not contain a full recognition sequence.

FIG. 8 shows the generation of 3′ overhangs in both the linearized plasmid and fragment (i.e., insert) by an exonuclease.

FIG. 9 shows the plasmid and fragment (i.e., insert) complementing and priming each other.

FIG. 10 shows a complete plasmid sequence after using a DNA polymerase and ligase. The lack of a PAM sequence and full target sequence prohibit Cas9 from working on (e.g., cutting) the newly completed, modified plasmid.

FIG. 11A-11D show aspects of the invention described herein. FIG. 11A shows the introduction of 1 exogenous nucleic acid sequence into a circular nucleic acid sequence. FIG. 11B shows the introduction of 2 exogenous nucleic acid sequences into a circular nucleic acid sequence. FIG. 11C shows the introduction of 3 exogenous nucleic acid sequences into a circular nucleic acid sequence. FIG. 11D shows the deletion of a region of a plasmid.

### DETAILED DESCRIPTION OF THE INVENTION

A description of example embodiments of the invention follows.

Described herein is the development of an efficient technology for the generation of novel cloning methods. Specifically, the clustered regularly interspaced short palindromic repeats (CRISPR) and CRISPR associated genes (Cas genes), referred to herein as the CRISPR/Cas system, has been adapted as an efficient cloning technology e.g., in combination with Gibson cloning. Demonstrated herein is that the CRISPR/Cas system can be used for the modification of one or more target nucleic acids.

Accordingly, in one aspect, the invention is directed to a method of modifying one or more target nucleic acid sequences. The method comprises contacting the one or more target nucleic acid sequences with (i) one or more ribonucleic acid (RNA) sequences wherein each RNA sequence comprises a portion that is complementary to all or a portion of one or more of the target nucleic acid sequences, (ii) a CRISPR associated (Cas) protein having nuclease activity, (iii) one or more exogenous nucleic acid sequences wherein at least one exogenous nucleic acid sequence comprises a 5′ adapter sequence that hybridizes to a 5′ flanking sequence of the target nucleic acid sequence and at least one exogenous nucleic acid sequence comprises a 3′ adapter sequence that hybridizes to a 3′ flanking sequence of the target nucleic acid sequence, and (iv) a nucleic acid sequence that binds a CRISPR associated protein, thereby producing a combination. The combination is maintained under conditions in which the one or more RNA sequences hybridize to all or the portion of the one or more target nucleic acid sequences to which each RNA sequence forms a complement thereby forming one or more base paired structures and the nucleic acid sequence that interacts with Cas protein directs Cas protein to cleave the one or more target nucleic acid sequences, thereby modifying the one or more target nucleic acid sequences.

As used herein, “modifying” (“modify”) one or more target nucleic acid sequences refers to changing all or a portion of a (one or more) target nucleic acid sequence and includes the cleavage, introduction (insertion), replacement, and/or deletion (removal) of all or a portion of a target nucleic acid sequence. All or a portion of a target nucleic acid sequence can be completely or partially modified using the methods provided herein. For example, modifying a target nucleic acid sequence includes replacing all or a portion of a target nucleic acid sequence with one or more nucleotides (e.g., an exogenous nucleic acid sequence) or removing or deleting all or a portion (e.g., one or more nucleotides) of a target nucleic acid sequence. Modifying the one or more target nucleic acid sequences also includes introducing or inserting one or more nucleotides (e.g., an exogenous sequence) into (within) one or more target nucleic acid sequences.

Modifying the one or more target nucleic acid sequence further includes a change to, or replacement of, one or more nucleotides of the one or more target nucleic acid sequences. For instance, a change can be a mutation (e.g., point, silent, missense, nonsense, insertion, deletion, etc.) to a target nucleic acid sequence. As will also be apparent to those of skill in the art, a change in one or more nucleotides in the target nucleic acid sequence can include a synonymous (conservative) substitution, a non-synonymous (non-conservative) substitution, or combination thereof.

As will be apparent to those of skill in the art, a variety of nucleic acid sequences can be targeted for modification. For example, the target nucleic acid sequence (the target nucleic acid sequence of interest) can be a single stranded nucleic acid, a double stranded nucleic acid or a combination thereof. The target nucleic acid sequence can comprise a plasmid, a plastid, a bacterial nucleic acid, a bacterial artificial chromosome, a viral nucleic acid, a mitochondrial nucleic acid, or an artificially synthesized nucleic acid. In a particular aspect, the target nucleic acid sequence comprises a circular nucleic acid sequence.

As will also be apparent to those of skill in the art, an (one or more) “exogenous” nucleic acid sequence refers to a sequence that is separate and distinct from the target nucleic acid sequence being modified.

In a particular aspect, the invention is directed to a method of introducing one or more exogenous nucleic acid sequences into one or more circular nucleic acid sequences. The method comprises contacting the one or more circular nucleic acid sequences with (i) one or more ribonucleic acid (RNA) sequences wherein each RNA sequence comprises a portion that is complementary to all or a portion of one or more target sequences within the one or more circular nucleic acid sequences, (ii) a CRISPR associated (Cas) protein having nuclease activity, (iii) one or more exogenous nucleic acid sequences wherein at least one exogenous nucleic acid sequence comprises a 5′ adapter sequence that hybridizes to a 5′ flanking sequence of the target nucleic acid sequence and at least one exogenous nucleic acid sequence comprises a 3′ adapter sequence that hybridizes to a 3′ flanking sequence of the target nucleic acid sequence; wherein at least one exogenous nucleic acid sequence comprises one or more additional nucleotides, and (iv) a nucleic acid sequence that interacts with Cas protein, thereby producing a combination. The combination is maintained under conditions in which the one or more RNA sequences hybridize to all or the portion of the one or more target nucleic acid sequences to which each RNA sequence forms a complement thereby forming one or more base paired structures and the nucleic acid sequence that interacts with Cas protein direct the Cas protein to cleave the target nucleic acid sequence, thereby introducing the one or more exogenous nucleic acid sequence into the one or more circular nucleic acid sequences.

The target nucleic acid sequence can be about 1 nucleotide, 2 nucleotides, 3 nucleotides, 4 nucleotides, 5 nucleotides, 10 nucleotides, 20 nucleotides, 50 nucleotides, 100 nucleotides, 200 nucleotides, 500 nucleotides, 1000 nucleotides, 2000 nucleotides or 5000 nucleotides. The target nucleic acid sequence can also be from about 1 nucleotide to about 5000 nucleotides, from about 2 nucleotides to about 2000 nucleotides, from about 3 nucleotides to about 1000 nucleotides, from about 4 nucleotides to about 500 nucleotides, from about 5 nucleotides to about 200 nucleotides, from about 10 nucleotides to about 100 nucleotides, or from about 20 nucleotides to about 50 nucleotides.

In some embodiments, a single target nucleic acid sequence is targeted. In other embodiments, more than one target nucleic acid sequence (e.g., 2, 3, 4, 5, 6, 7, 8, 9, 10 sequences) is targeted. In some embodiments, the target nucleic sequence or sequences can be a contiguous sequence. In other embodiments, the target nucleic sequence or sequences can be non-contiguous sequences.

Non-contiguous target nucleic acid sequences may comprise one or more linker sequences. As used herein, a “linker” is something that connects two or more nucleic acid or amino acid sequences. As will be appreciated by one of ordinary skill in the art, a variety of linkers can be used (e.g., Greg T. Hermanson, Bioconjugate Techniques, Academic Press 1996).

In the methods provided herein, the one or more target nucleic acid sequences is contacted with one or more ribonucleic acid (RNA) sequences that comprise a portion that is complementary to all or a portion of one or more target nucleic acid sequences. As used herein, the RNA sequence is sometimes referred to as guide RNA (gRNA) or single guide RNA (sgRNA). See, for example, U.S. Pat. Nos. 8,697,359 and 8,771,945 which are incorporated herein by reference.

In some aspects, the (one or more) RNA sequence can be complementary to one or more (e.g., some; all) of the target nucleic acid sequences that are being modified. In one aspect, the RNA sequence is complementary to all or a portion of a single target nucleic acid sequence. In a particular aspect in which two or more target nucleic acid sequences are to be modified, multiple (e.g., 2, 3, 4, 5, 6, 7, 8, 9, 10, or more than 10) RNA sequences can be introduced wherein each RNA sequence is complementary to or specific for all or a portion of at least one target nucleic acid sequence. In some aspects, two or more, three or more, four or more, five or more, or six or more, etc., RNA sequences are complementary to (specific for) different parts of the same target sequence. In one aspect, two or more RNA sequences bind to different sequences of the same region (e.g. promoter) of target nucleic acid. In some aspects, a single RNA sequence is complementary to at least two target or more (every; all) of the target nucleic acid sequences. It will also be apparent to those of skill in the art that the portion of the RNA sequence that is complementary to one or more of the target nucleic acid sequences and the nucleic acid sequence comprising a CRISPR associated protein binding site can be introduced as a single sequence or as 2 (or more) separate sequences.

In some aspects, the RNA sequence used to hybridize to a target nucleic acid sequence is a naturally occurring RNA sequence, a modified RNA sequence (e.g., a RNA sequence comprising one or more modified bases), a synthetic RNA sequence, or a combination thereof. As used herein a “modified RNA” is an RNA comprising one or more modifications (e.g., RNA comprising one or more non-standard and/or non-naturally occurring bases) to the RNA sequence (e.g., modifications to the backbone and or sugar). Methods of modifying bases of RNA are well known in the art. Examples of such modified bases include those contained in the nucleosides 5-methylcytidine (5mC), pseudouridine (T), 5-methyluridine, 2′O-methyluridine, 2-thiouridine, N-6 methyladenosine, hypoxanthine, dihydrouridine (D), inosine (I), and 7-methylguanosine (m7G). It should be noted that any number of bases in a RNA sequence can be substituted in various embodiments. It should further be understood that combinations of different modifications may be used.

In some aspects, the RNA sequence is a morpholino. Morpholinos are typically synthetic molecules, of about 25 bases in length and bind to complementary sequences of RNA by standard nucleic acid base-pairing. Morpholinos have standard nucleic acid bases, but those bases are bound to morpholine rings instead of deoxyribose rings and are linked through phosphorodiamidate groups instead of phosphates. Morpholinos do not degrade their target RNA molecules, unlike many antisense structural types (e.g., phosphorothioates, siRNA). Instead, morpholinos act by steric blocking and bind to a target sequence within a RNA and block molecules that might otherwise interact with the RNA.

Each RNA sequence can vary in length from about 10 base pairs (bp) to about 200 bp. In some embodiments, the RNA sequence can be about 11 to about 190 bp; about 12 to about 150 bp; about 15 to about 120 bp; about 20 to about 100 bp; about 30 to about 90 bp; about 40 to about 80 bp; about 50 to about 70 bp in length.

The portion of each target nucleic acid sequence to which each RNA sequence is complementary can also vary in length. In particular aspects, the portion of each target nucleic acid sequence to which the RNA is complementary can be about 8, 9, 10, 11, 12, 13, 14, 15, 16, 17, 18, 19, 20, 21, 22, 23, 24, 25, 26, 27, 28, 29, 30, 31, 32, 33, 34,35, 36, 37, 38 39, 40, 41, 42, 43, 44, 45, 46 47, 48, 49, 50, 51, 52, 53,54, 55, 56,57, 58, 59 60, 61, 62, 63, 64, 65, 66, 67, 68, 69, 70, 71, 72, 73, 74, 75, 76, 77, 78, 79, 80 81, 82, 83, 84, 85, 86, 87 88, 89, 90, 81, 92, 93, 94, 95, 96, 97, 98, or 100 nucleotides (e.g., contiguous nucleotides; non-contiguous nucleotides) in length. In some embodiments, each RNA sequence can be at least about 70%, 75%, 80%, 85%, 90%, 95%, 100%, etc. identical or similar to all or a portion of each target nucleic acid sequence. In some embodiments, each RNA sequence is completely or partially identical or similar to one or more target nucleic acid sequence. For example, each RNA sequence can differ from perfect complementarity to the portion of the target sequence by about 1, 2, 3, 4, 5, 6, 7, 8, 9, 10, 11, 12, 13, 14, 15, 16, 17, 18, 19, 20, etc. nucleotides. In some embodiments, one or more RNA sequences are perfectly complementary (100%) across at least about 10 to about 25 (e.g., about 20) nucleotides of the target nucleic acid.

In the methods provided herein, the one or more target nucleic acid sequences are contacted with one or more CRISPR associated (Cas) proteins having nuclease activity (e.g., RNA-guided (gRNA) nuclease activity). See, for example, U.S. Pat. Nos. 8,697,359 and 8,771,945 which are incorporated herein by reference.

Bacteria and Archaea have evolved an RNA-based adaptive immune system that uses CRISPR (clustered regularly interspaced short palindromic repeat) and Cas (CRISPR-associated) proteins to detect and destroy invading viruses and plasmids (Horvath and Barrangou, Science, 327(5962):167-170 (2010); Wiedenheft et al., Nature, 482(7385):331-338 (2012)). Cas proteins, CRISPR RNAs (crRNAs) and trans-activating crRNA (tracrRNA) form ribonucleoprotein complexes, which target and degrade specific foreign nucleic acids, guided by crRNAs (Gasiunas et al., Proc. Natl. Acad. Sci, 109(39):E2579-86 (2012); Jinek et al., Science, 337:816-821 (2012)). The components of this system are used in the methods described herein and include a guide RNA (gRNA) and a CRISPR associated nuclease (e.g., Cas9). The gRNA/Cas9 complex can be recruited to a target sequence by the base-pairing between the gRNA and the target sequence. Binding of Cas9 to the target sequence also requires the correct Protospacer Adjacent Motif (PAM) sequence adjacent to the target sequence. The binding of the gRNA/Cas9 complex localizes the Cas9 to the target nucleic acid sequence so that the Cas9 can cut both strands of nucleic acid (e.g., DNA).

In particular aspects an appropriate Cas protein may be selected such that the target nucleic acid sequence will contain a PAM for that particular Cas protein at an appropriate position. For example, if the target nucleic acid sequence does not contain a PAM for Streptococcus pyogenes Cas9 within an appropriate portion of the target nucleic acid sequence, then an alternate Cas protein for which the target nucleic acid does contain an appropriately positioned PAM sequence may be used.

One or more Cas proteins or variants thereof cleave each of the target nucleic acid sequences. Any variant of Cas9 that retains RNA guided nuclease activity can be used in the methods of the invention. In some aspects, the Cas nucleic acid sequence encodes a Cas9 protein that comprises one or more mutations. In some aspects, the Cas nucleic acid sequence encodes a Cas9 protein that comprises a mutation at amino acid position 10, 840, or a combination thereof. In some aspects, the Cas nucleic acid sequence encodes a Cas9 protein wherein the amino acid at position 10 is mutated from aspartate (D) to alanine (A) and/or the amino acid at position 840 is mutated from histidine (H) to alanine (A).

A variety of CRISPR associated (Cas) genes or proteins which are known in the art can be used in the methods of the invention and the choice of Cas protein will depend upon the particular conditions of the method (e.g., www.ncbi.nlm.nih.gov/gene/?term=cas9, U.S. Pat. Nos. 8,697,359 and 8,771,945 which are incorporated herein by reference. Specific examples of Cas proteins include Cas1, Cas2, Cas3, Cas4, Cas5, Cas6, Cas7, Cas8, Cas9 and Cas10. In a particular aspect, the Cas nucleic acid or protein used in the methods is Cas9. In some embodiments a Cas protein, e.g., a Cas9 protein, may be from any of a variety of prokaryotic species. In some embodiments a particular Cas protein, e.g., a particular Cas9 protein, may be selected to recognize a particular protospacer-adjacent motif (PAM) sequence. In certain embodiments a Cas protein, e.g., a Cas9 protein, may be obtained from a bacteria or archaea or synthesized using known methods. In certain embodiments, a Cas protein may be from a gram positive bacteria or a gram negative bacteria. In certain embodiments, a Cas protein may be from a Streptococcus, (e.g., a S. pyogenes (Accession No. Q99ZW2), a S. thermophiles (Accession No. G3ECR1)) a Cryptococcus, a Corynebacterium, a Haemophilus, a Eubacterium, a Pasteurella, a Prevotella, a Veillonella, or a Marinobacter. In some embodiments nucleic acids encoding two or more different Cas proteins, or two or more Cas proteins, may be used, e.g., to allow for recognition and modification of sites comprising the same, similar or different PAM motifs.

In the methods provided herein, the one or more target nucleic acids are contacted with a (one or more) nucleic acid sequence that interacts (complexes; binds) with a (one or more) Cas protein (a Cas interacting sequence). See, for example, U.S. Pat. Nos. 8,697,359 and 8,771,945 which are incorporated herein by reference. Nucleic acid sequences that interact with Cas protein and that along with based paired RNA structures direct Cas protein to deplete targeted sequences, are known in the art (e.g., see Jinek et al., Science, 337:816-821 (2012); Cong et al., Science, 339:819-823 (2013); Ran et al., Nature Protocols, 8(11):2281-2308 (2013); Mali et al., Sciencexpress, 1-5 (2013) all of which are incorporated herein by reference). In some aspects, such nucleic acid sequences are referred to as trans-activating CRISPR nucleic acid. In one aspect, the nucleic acid that interacts with Cas protein is an RNA sequence (sometimes referred to as trcrRNA). In other aspects, the nucleic acid sequence that interacts with a Cas protein can also hybridize to all or a portion of one or more of the RNA sequences that are complementary to all or a portion of at least one target sequence. In a particular aspect, the nucleic acid sequence that interacts with a Cas protein does not hybridize to all or the same portion of the RNA sequence that is complementary to all or a portion of at least one target sequence.

In one aspect, the one or more RNA sequences and the one or more nucleic acid sequences that interacts with the Cas protein are included as a single (the same) nucleic acid sequence. In another aspect, the nucleic acid sequence that interacts with the Cas protein is introduced as one or more separate nucleic acid sequences (e.g., not included in one, more or all of the one or more RNA sequences). In a particular aspect, upon hybridization of the one or more RNA sequences to the one or more target nucleic acids thereby forming one or more base paired structures, the one or more base paired structures and the nucleic acid sequence that interacts with the Cas protein direct the Cas protein or variants thereof to cleave the one or more target nucleic acids sequences.

The methods described herein can further comprise assessing whether the one or more target nucleic acids have been modified using a variety of known methods, e.g., sequencing. As will be appreciated by one of skill in the art, known methods of DNA sequencing include chemical sequencing, chain-termination methods, de novo sequencing and others. In some embodiments assessing whether the one or more target nucleic acids have been modified comprises performing a restriction enzyme digest on the target nucleic acid. For example, if the modification introduced or removed a restriction site for a particular restriction enzyme, such modification may be detected by performing a restriction digest using the enzyme and analyzing the resulting restriction fragments by gel electrophoresis.

As described herein, the one or more target nucleic acid sequences to be modified are contacted with one or more exogenous nucleic acid sequences, wherein at least one exogenous nucleic acid sequence comprises a 5′ adapter sequence that hybridizes to a 5′ flanking sequence of the target nucleic acid sequence and at least one exogenous nucleic acid sequence comprises a 3′ adapter sequence that hybridizes to a 3′ flanking sequence of the target nucleic acid sequence.

As used herein, “adapter sequence” refers to a nucleic acid sequence that can bind to or hybridize to a nucleic acid sequence. In one aspect, the adapter sequences binds to or hybridizes to a target nucleic acid sequence. In another aspect, the adapter sequences binds to or hybridizes to a flanking sequence of the target nucleic acid. As is apparent to those of skill in the art, a “flanking sequence” of a target nucleic acid sequence refers to a sequence that is 5′ and/or 3′ of the target nucleic acid sequence. In one aspect, a target nucleic acid sequence comprises a 5′ flanking sequence. In another aspect, a target nucleic acid sequence comprises a 3′ flanking sequence. In yet another aspect, a target nucleic acid sequence comprises a 5′ and a 3′ flanking sequence.

The 5′ and/or 3′ adapter sequence can completely or partially hybridize to a 5′ and/or 3′ flanking sequence of the target nucleic acid sequence. In one aspect, a 3′ adapter sequence completely or partially hybridizes to a 3′ flanking sequence of the target nucleic acid sequence. In another aspect, the 5′ adapter sequence completely or partially hybridizes to a 5′ flanking sequence of the target nucleic acid sequence. In another aspect, the exogenous nucleic acid comprises a 3′ adapter sequence. In yet another embodiment, the exogenous nucleic acid comprises both a 5′ and a 3′ adapter sequence. In some aspects, one or more adapter sequence comprises a (one or more) PAM sequence, e.g., to avoid formation of an adapter concatamer

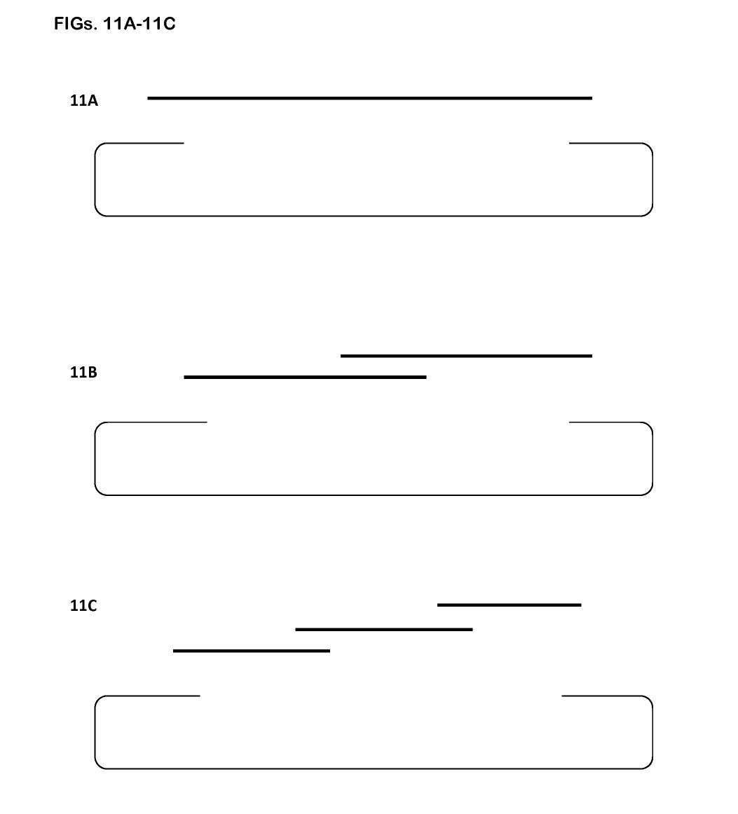

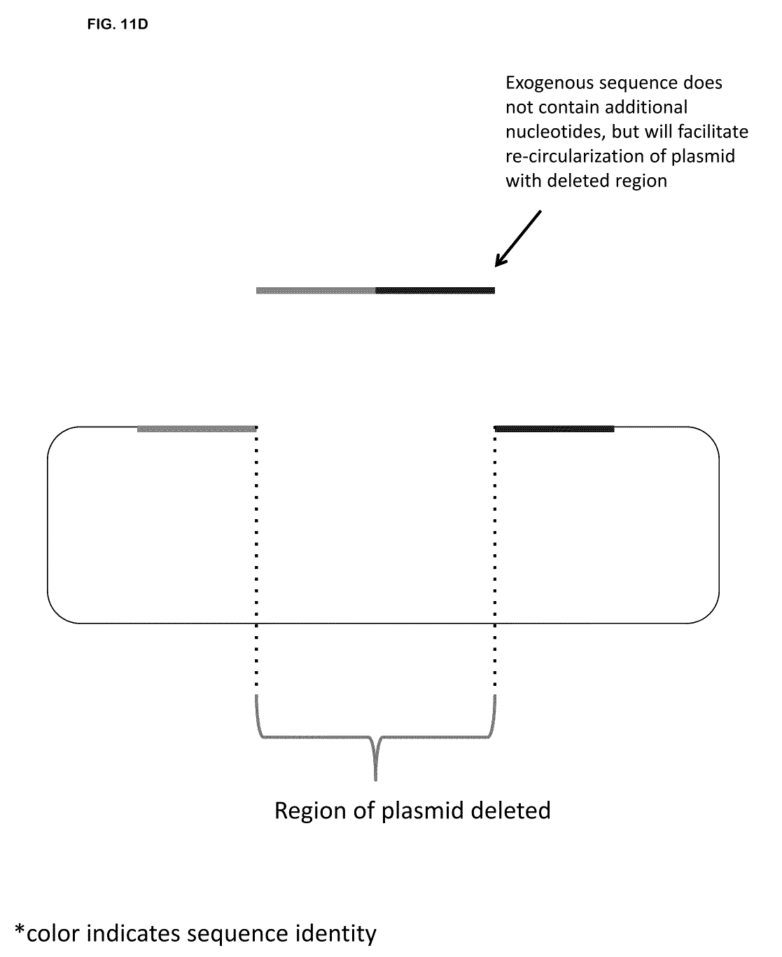

In another aspect, the adapter sequence binds to or hybridizes to all or a portion of one or more exogenous nucleic acid sequences. A (one or more) adapter sequence of an (one or more) exogenous nucleic acid sequence can completely or partially bind to or hybridize to an adapter sequence of another exogenous nucleic acid sequence. For example, a 3′ adapter sequence of one exogenous nucleic acid sequence can bind or hybridize to a 3′ adapter sequence of another exogenous nucleic acid sequence. Similarly, a 5′ adapter sequence of one exogenous nucleic acid sequence can bind or hybridize to a 5′ adapter sequence of another exogenous nucleic acid sequence. In these instances, two or more exogenous nucleic acid sequences modify one or more target nucleic acid sequences (see e.g., FIG. 11).

As will be appreciated by those of skill in the art, the length of the adapter sequence can vary. In some aspects, the adapter sequence is about 1 nucleotide to about 100 nucleotides in length. In some aspects, the adapter sequence is about 10 nucleotides to about 100 nucleotides in length. In other embodiments, the adapter sequence is about 5 nucleotides to about 80 nucleotides. In other embodiments, the adapter sequence is about 10 nucleotides to about 60 nucleotides. In other embodiments, the adapter sequence is about 15 nucleotides to about 40 nucleotides. In other embodiments, the adapter sequence is about 20 nucleotides to about 30 nucleotides. In some embodiments, the adapter sequence is less than 10 nucleotides (e.g., 1, 2, 3, 4, 5, 6, 7, 8, 9). In other embodiments, the adapter sequence is greater than 100 nucleotides.

In some aspects, one or more exogenous nucleic acid sequences can further comprise one or more additional nucleotides (e.g., an additional nucleic acid sequence) either 5′ or 3′ of the adapter sequence. In aspects in which an exogenous nucleic acid sequence comprises a 5′ adapter and a 3′ adapter, the one or more additional nucleotides can be in between the 5′ adapter and the 3′ adapter. In some aspects, the one or more additional nucleotides can be a single base or multiple bases (e.g., a nucleic acid sequence).

As will be apparent to those of skill in the art, the one or more additional nucleotides can comprise 1, 2, 3, 4, 5, 6, 7, 8, 9, 10, more than 10, more than 20, more than 30, more than 40, more than 50, more than 100, more than 200, more than 300, more than 400, more than 500, more than 1000, more than 2000, more than 3000 nucleotides.

As will be apparent to those of skill in the art, the one or more additional nucleotides can be, for example, a nucleotide variant, a restriction site, a cloning site, a recombination site and portions or combinations thereof.

In a particular aspect of the invention, the one or more additional nucleotides comprise a gene. In another aspect of the invention, the one or more additional nucleotides comprise a portion of a gene. The portion of a gene can comprise an exon, an intron, a 5′ untranslated region, a 3′ untranslated region, portions thereof, or combinations thereof.

In another aspect of the invention, the one or more additional nucleotides comprise a regulatory sequence. Examples of a regulatory sequence include a promoter sequence, an enhancer sequence, a TATA box, a repressor sequence, an insulator sequence, a terminator signal, a sequence targeted for epigenetic modification, portions thereof, or combinations thereof.

In another aspect of the invention, the one or more additional nucleotides encode a RNA sequence. Examples of RNA sequences include an internal ribosome entry site (IRES), a MS2 tag, a riboswitch, a RNA affinity purification sequence, a RNA localization signal, a non-coding RNA sequence, a RNA binding site, shRNA, miRNA precursor, portions thereof, or combinations thereof.

In yet another aspect of the invention, the additional nucleotides comprise a nucleic acid sequence that encodes a polypeptide. Examples of polypeptides include a tag, a transcription factor, an enzyme, a cytokine, a receptor, a transporter, a secreted protein, a binding protein, a post-translational modifying protein, a post-transcriptional modifying protein, a cytoskeletal protein, portions thereof, or combinations thereof.

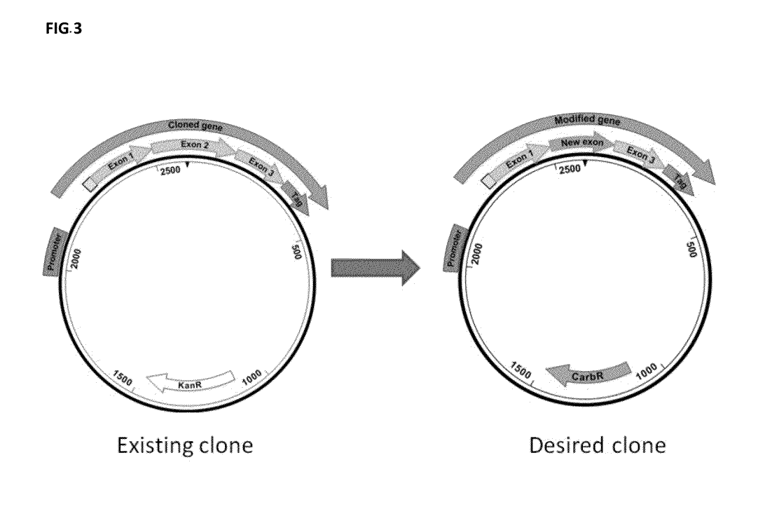

In some aspects a target nucleic acid sequence to be modified encodes a tag. The exogenous nucleic acid sequence may be inserted into a target nucleic acid sequence (e.g., a plasmid) in an appropriate position such that a protein comprising the tag is produced (e.g., see FIG. 3). The term “tag” is used in a broad sense to encompass any nucleic acid sequence that encodes any of a wide variety of polypeptides or refers to the polypeptides themselves. In some aspects, a tag comprises a sequence useful for purifying, expressing, solubilizing, and/or detecting a polypeptide. In some aspects, a tag may serve multiple functions. In some aspects, a tag is a relatively small polypeptide, e.g., ranging from a few amino acids up to about 100 amino acids long. In some embodiments a tag is more than 100 amino acids long, e.g., up to about 500 amino acids long, or more. In some aspects, the tag is an antibiotic marker, a fluorescent protein, a selection marker, a protein stabilizing signal, a protein de-stabilizing signal, a degron, a degradation signal, a secretion sequence signal, a nuclear localization signal, an amino acid sequence for immunoprecipitation, an amino acid sequence for affinity purification, a protein localization sequence, portions thereof, or combinations thereof. In some aspects, a tag comprises an HA, TAP, Myc, 6× His, Flag, V5, or GST tag, to name few examples. A tag (e.g., any of the afore-mentioned tags) that comprises an epitope against which an antibody, e.g., a monoclonal antibody, is available (e.g., commercially available) or known in the art may be referred to as an “epitope tag”. In some aspects a tag comprises a solubility-enhancing tag (e.g., a SUMO tag, NUS A tag, SNUT tag, a Strep tag, or a monomeric mutant of the Ocr protein of bacteriophage T7). See, e.g., Esposito D and Chatterjee D K. Curr Opin Biotechnol.; 17(4):353-8 (2006). In some aspects, a tag is cleavable, so that at least a portion of it can be removed, e.g., by a protease. In some aspects, this is achieved by including a protease cleavage site in the tag, e.g., adjacent or linked to a functional portion of the tag. Exemplary proteases include, e.g., thrombin, TEV protease, Factor Xa, PreScission protease, etc. In some aspects, a “self-cleaving” tag is used. See, e.g., PCT/US05/05763. In some aspects, a tag comprises a fluorescent polypeptide (e.g., GFP or a derivative thereof such as enhanced GFP (EGFP)) or an enzyme that can act on a substrate to produce a detectable signal, e.g., a fluorescence or colorimetric signal. Luciferase (e.g., a firefly, Renilla, or Gaussia luciferase) is an example of such an enzyme. Examples of fluorescent proteins include GFP and derivatives thereof, proteins comprising chromophores that emit light of different colors such as red, yellow, and cyan fluorescent proteins, etc. A tag, e.g., a fluorescent protein, may be monomeric. In certain aspects, a fluorescent protein is e.g., Sirius, Azurite, EBFP2, TagBFP, mTurquoise, ECFP, Cerulean, TagCFP, mTFP1, mUkG1, mAG1, AcGFP1, TagGFP2, EGFP, mWasabi, EmGFP, TagYPF, EYFP, Topaz, SYFP2, Venus, Citrine, mKO, mKO2, mOrange, mOrange2, TagRFP, TagRFP-T, mStrawberry, mRuby, mCherry, mRaspberry, mKate2, mPlum, mNeptune, mTomato, T-Sapphire, mAmetrine, mKeima. See, e.g., Chalfie, M. and Kain, S R (eds.) Green fluorescent protein: properties, applications, and protocols (Methods of biochemical analysis, v. 47). Wiley-Interscience, Hoboken, N.J., 2006, and/or Chudakov, D M, et al., Physiol Rev. 90(3):1103-63, 2010 for discussion of GFP and numerous other fluorescent or luminescent proteins. In some aspects, a tag may comprise a domain that binds to and/or acts a sensor of a small molecule (e.g., a metabolite) or ion, e.g., calcium, chloride, or of intracellular voltage, pH, or other conditions. Any genetically encodable sensor may be used; a number of such sensors are known in the art. In some aspects a FRET-based sensor may be used. In some aspects different target nucleic acids (e.g., genes) are modified to incorporate different tags, so that proteins encoded by the genes are distinguishably labeled. For example, between 2 and 20 distinct tags may be introduced. In some aspects the tags have distinct emission and/or absorption spectra. In some aspects a tag may absorb and/or emit light in the infrared or near-infrared region. It will be understood that any nucleic acid sequence encoding a tag may be codon-optimized for expression in a biological system (e.g., a cell, bacteria, zygote, embryo, or animal) into which it is to be introduced.

In some aspects a target nucleic acid sequence comprises one or more fragments or domains of a protein, which when modified using the methods provided herein may act in a dominant negative manner and may, for example, disrupt normal function or interaction of the protein.

In some aspects a target nucleic acid sequence (e.g., a gene) encodes a protein the aggregation of which is associated with one or more diseases, such as protein misfolding diseases. Examples of such proteins include, e.g., alpha-synuclein (Parkinson's disease and related disorders), amyloid beta or tau (Alzheimer's disease), TDP-43 (frontotemporal dementia, ALS).

In some aspects a target nucleic acid sequence (e.g., a gene) encodes a transcription factor, a transcriptional co-activator or co-repressor, an enzyme, a chaperone, a heat shock factor, a heat shock protein, a receptor, a secreted protein, a transmembrane protein, a histone (e.g., H1, H2A, H2B, H3, H4), a peripheral membrane protein, a soluble protein, a nuclear protein, a mitochondrial protein, a growth factor, a cytokine (e.g., an interleukin, e.g., any of IL-1-IL-33), an interferon (e.g., alpha, beta, or gamma), a chemokine (e.g., a CXC, CX3C, C (or XC), or CX3C chemokine) A chemokine may be CCL1-CCL28, CXCL1-CXCL17, XCL1 or XCL2, or CXC3L1). In some aspects a target nucleic acid sequence encodes a colony-stimulating factor, a hormone (e.g., insulin, thyroid hormone, growth hormone, estrogen, progesterone, testosterone), an extracellular matrix protein (e.g., collagen, fibronectin), a motor protein (e.g., dynein, myosin), cell adhesion molecule, a major or minor histocompatibility (MHC) gene, a transporter, a channel (e.g., an ion channel), an immunoglobulin (Ig) superfamily (IgSF) gene (e.g., a gene encoding an antibody, T cell receptor, B cell receptor), tumor necrosis factor, an NF-kappaB protein, an integrin, a cadherin superfamily member (e.g., a cadherin), a selectin, a clotting factor, a complement factor, a plasminogen, plasminogen activating factor. Growth factors include, e.g., members of the vascular endothelial growth factor (VEGF, e.g., VEGF-A, VEGF-B, VEGF-C, VEGF-D), epidermal growth factor (EGF), insulin-like growth factor (IGF; IGF-1, IGF-2), fibroblast growth factor (FGF, e.g., FGF1-FGF22), platelet derived growth factor (PDGF), or nerve growth factor (NGF) families. It will be understood that the afore-mentioned protein families comprise multiple members. Any such member may be used in various aspects. In some aspects a growth factor promotes proliferation and/or differentiation of one or more hematopoietic cell types. For example, a growth factor may be CSF1 (macrophage colony-stimulating factor), CSF2 (granulocyte macrophage colony-stimulating factor, GM-CSF), or CSF3 (granulocyte colony-stimulating factors, G-CSF). In some aspects a gene encodes erythropoietin (EPO). In some aspects, a target nucleic acid sequence encodes a neurotrophic factor, i.e., a factor that promotes survival, development and/or function of neural lineage cells (which term as used herein includes neural progenitor cells, neurons, and glial cells, e.g., astrocytes, oligodendrocytes, microglia). For example, in some embodiments, the protein is a factor that promotes neurite outgrowth. In some aspects, the protein is ciliary neurotrophic factor (CNTF) or brain-derived neurotrophic factor (BDNF).

In some aspects a target nucleic acid sequence (e.g., a gene) encodes a polypeptide that is a subunit of a protein that is comprised of multiple subunits.

In other aspects, the target nucleic acid sequence encodes an enzyme. An enzyme may be any protein that catalyzes a reaction of a type that has been assigned an Enzyme Commission number (EC number) by the Nomenclature Committee of the International Union of Biochemistry and Molecular Biology (NC-IUBMB). Enzymes include, oxidoreductases, transferases, hydrolases, lyases, isomerases, and ligases. Examples include, e g , kinases (protein kinases, e.g., Ser/Thr kinase, Tyr kinase), lipid kinases (e.g., phosphatidylinositide 3-kinases (PI 3-kinases or PI3Ks)), phosphatases, acetyltransferases, methyltransferases, deacetylases, demethylases, lipases, cytochrome P450s, glucuronidases, recombinases (e.g., Rag-1, Rag-2). An enzyme may participate in the biosynthesis, modification, or degradation of nucleotides, nucleic acids, amino acids, proteins, neurotransmitters, xenobiotics (e.g., drugs) or other macromolecules.

In a particular aspect, the target nucleic acid sequence encodes a kinase. The mammalian genome encodes at least about 500 different kinases. Kinases can be classified based on the nature of their typical substrates and include protein kinases (i.e., kinases that transfer phosphate to one or more protein(s)), lipid kinases (i.e., kinases that transfer a phosphate group to one or more lipid(s)), nucleotide kinases, etc. Protein kinases (PKs) are of particular interest in certain aspects of the invention. PKs are often referred to as serine/threonine kinases (S/TKs) or tyrosine kinases (TKs) based on their substrate preference. Serine/threonine kinases (EC 2.7.11.1) phosphorylate serine and/or threonine residues while TKs (EC 2.7.10.1 and EC 2.7.10.2) phosphorylate tyrosine residues. A number of “dual specificity” kinases (EC 2.7.12.1) that are capable of phosphorylating both serine/threonine and tyrosine residues are known. The human protein kinase family can be further divided based on sequence/structural similarity into the following groups: (1) AGC kinases—containing PKA, PKC and PKG; (2) CaM kinases—containing the calcium/calmodulin-dependent protein kinases; (3) CK1—containing the casein kinase 1 group; (4) CMGC—containing CDK, MAPK, GSK3 and CLK kinases; (5) STE—containing the homologs of yeast Sterile 7, Sterile 11, and Sterile 20 kinases; (6) TK—containing the tyrosine kinases; (7) TKL—containing the tyrosine-kinase like group of kinases. A further group referred to as “atypical protein kinases” contains proteins that lack sequence homology to the other groups but are known or predicted to have kinase activity, and in some instances are predicted to have a similar structural fold to typical kinases.

In another aspect, the target nucleic acid sequence encodes a receptor. Receptors include, e.g., G protein coupled receptors, tyrosine kinase receptors, serine/threonine kinase receptors, Toll-like receptors, nuclear receptors, immune cell surface receptors. In some embodiments a receptor is a receptor for any of the hormones, cytokines, growth factors, or secreted proteins mentioned herein. Numerous G protein coupled receptors (GPCRs) are known in the art. See, e.g., Vroling B, GPCRDB: information system for G protein-coupled receptors. Nucleic Acids Res. 2011 January; 39(Database issue):D309-19. Epub 2010 Nov. 2. The GPCRDB can be found online at http://www.gper.org/7tm/. G protein coupled receptors include, e.g., adrenergic, cannabinoid, purinergic receptors, neuropeptide receptors, olfactory receptors. Transcription factors (TFs) (sometimes called sequence-specific DNA-binding factors) bind to specific DNA sequences and (alone or in a complex with other proteins), regulate transcription, e.g., activating or repressing transcription. Exemplary TFs are listed, for example, in the TRANSFAC® database, Gene Ontology (http://www.geneonlology.org/) or DBD (www.transcriptionfactor.org) (Wilson, et al, DBD—taxonomically broad transcription factor predictions: new content and functionality Nucleic Acids Research 2008 doi:10.1093/nar/gkm964). TFs can be classified based on the structure of their DNA binding domains (DBD). For example in certain embodiments a TF is a helix-loop-helix, helix-turn-helix, winged helix, leucine zipper, bZIP, zinc finger, homeodomain, or beta-scaffold factor with minor groove contacts protein. Transcription factors include, e.g., p53, STAT3, PAS family transcription factors (e.g., HIF family: HIF1A, HIF2A, HIF3A), aryl hydrocarbon receptor.

In some aspects it may be of interest to modify multiple target nucleic acid sequences that function in the same biological pathway or process, e.g., signal transduction pathway, biosynthetic pathway, xenobiotic metabolizing pathway, anabolic or catabolic pathway, apoptosis, autophagy, endocytosis, exocytosis. In some aspects the modification of one or more target nucleic acid sequences according to inventive methods is useful for studying drug metabolism. For example, it may be of interest to modify multiple enzymes involved in xenobiotic metabolism (e.g., multiple P450s). In some aspects, the modification of one or more target nucleic acid sequences according to inventive methods is useful for studying the immune system and/or for generating animals that have a humanized immune system or that are immunocompromised and may serve as hosts for cells or tissues from other organisms of the same species or different species.

In another aspect of the invention, the methods of modifying a target nucleic acid sequence can comprise contacting the combination with one or more exonucleases, polymerases and ligases. As will be appreciated by one of skill in the art, an exonuclease is an enzyme that cleaves nucleotides from the end of a polynucleotide chain. In some embodiments, the one or more exonucleases is a 5′ exonuclease, a 3′ exonuclease, or a combination thereof. In another embodiment, the exonuclease is a prokaryotic exonuclease or a eukaryotic exonuclease. In some embodiments, the exonuclease is exonuclease I, II, III, IV, V, or VIII. One of skill in the art will also appreciate a polymerase is an enzyme that synthesizes nucleic acid polymers. In some embodiments, the one or more polymerases is a DNA polymerase, a RNA polymerase, or a combination thereof. In some embodiments the polymerase is a DNA polymerase that has 3′→5′ exonuclease activity that mediates proofreading. One of skill in the art will appreciate a ligase is an enzyme that can join nucleic acid strands together. In some embodiments, the one or more ligases is a DNA ligase. Examples of DNA ligases include the E. Coli DNA ligase, T4 DNA ligase (from bacteriophage T4), mammalian ligases, and thermostable ligases (from thermophilic bacteria). In another embodiment, the one or more ligases is a RNA ligase. Examples of RNA ligases include the E. Coli RNA ligase 1 (ssRNA ligase). In some embodiments the exonuclease is a 5′ DNA exonuclease such as T5 exonuclease, the polymerase is a DNA polymerase such as Phusion® DNA polymerase, and the ligase is a DNA ligase, e.g., Taq DNA ligase. In some embodiments, e.g., if the nucleic acid to be modified is DNA, the exonuclease, polymerase and ligase may be a DNA exonuclease, DNA polymerase, and DNA ligase. The composition in which the nucleic acid modification reaction is performed may comprise nucleotides (e.g., dGTP, dATP, dTTP, dCTP—presumably these are needed for the polymerase) and any cofactors (e.g., metal ions, e.g., that may be needed for activity of any of the enzymes).

As described herein, the one or more target nucleic acid sequences to be modified are contacted with one or more RNA sequences, a Cas protein, one or more exogenous nucleic acid sequences, and a nucleic acid sequence that interacts with Cas binding, thereby producing a combination. The combination is maintained under conditions in which the one or more RNA sequences hybridize to all or a portion of the one or more target nucleic acid sequences to which each RNA sequence forms a complement thereby forming one or more base paired structures, and the one or more base paired structures and the nucleic acid sequence that interacts with Cas protein direct Cas protein to cleave the one or more target nucleic acid sequences (e.g., by forming a complex (a CRISPR complex)), thereby modifying the one or more target nucleic acid sequences. See, for example, U.S. Pat. Nos. 8,697,359 and 8,771,945 which are incorporated herein by reference.

In some aspects of the invention, the method of modifying a target nucleic acid sequence can comprise contacting the target nucleic acid sequence with the one or more RNA sequences, the Cas protein, the one or more exogenous nucleic acid sequences and the nucleic acid sequence that interacts with Cas protein in any order. In one aspect, the method can comprise contacting the target nucleic acid sequence with one or more RNA sequences, the Cas protein, the one or more exogenous nucleic acid sequence and the nucleic acid sequence that interacts with Cas protein simultaneously. In another aspect, the method can comprise contacting the target nucleic acid sequence with the one or more RNA sequences, the Cas protein, the one or more exogenous nucleic acid sequences and the nucleic acid sequence that interacts with Cas protein sequentially. In yet another aspect, the nucleic acid sequence comprising a Cas protein binding site can be added simultaneously or sequentially with the other components (producing a combination). As will be appreciated by one of skill in the art, the components of the combination and the methods described herein can be combined using known lab techniques and known solutions (e.g., buffers).

In some aspects of the invention, the method of modifying one or more target nucleic acids comprises maintaining the combination in an isothermal condition (e.g., at 37° C.). In some aspects, the method of modifying one or more target nucleic acids comprises maintaining the combination near isothermal conditions. In some aspects the combination is maintained or performed at a range of temperatures (e.g., 0-100° C., 4-10° C., 37-95° C.) or at two or more different temperatures (e.g., at 37° C. and then at 50° C.). It will be appreciated by one of skill in the art at which optimal temperature or temperatures are appropriate to maintain the combination.

Combinations and compositions described herein are aspects of the invention. For example, in some aspects, the invention provides a composition comprising: (i) one or more ribonucleic acid (RNA) sequences wherein each RNA sequence comprises a portion that is complementary to all or a portion of one or more of the target nucleic acid sequences, (ii) a (one or more) CRISPR associated (Cas) protein having nuclease activity (e.g., a Cas9 protein), (iii) one or more exogenous nucleic acid sequences wherein at least one exogenous nucleic acid sequence comprises a 5′ adapter sequence that hybridizes to a 5′ flanking sequence of the target nucleic acid sequence and at least one exogenous nucleic acid sequence comprises a 3′ adapter sequence that hybridizes to a 3′ flanking sequence of the target nucleic acid sequence, and (iv) a nucleic acid sequence that interacts with Cas protein. In some embodiments, the composition further comprises an exonuclease, a polymerase, and/or a ligase, e.g., an exonuclease, a polymerase, and a ligase. In various embodiments the RNA sequence(s), Cas protein having nuclease activity, exogenous nucleic acid sequence(s), nucleic acid sequence that interacts with Cas protein, exonuclease, polymerase, and ligase may be any of those described herein and may have any of the properties described herein.

In some aspects, a nucleic acid that has been modified or generated as described herein (e.g., that comprises a modification generated as described herein) may be subjected to additional manipulations and/or used for any of a variety of purposes. For example, a nucleic acid that has been modified or generated as described herein may be subjected to amplification (e.g., by PCR or rolling circle amplification), in vitro transcription, or in vitro translation of at least a portion of the nucleic acid.

In some embodiments a nucleic acid that has been modified or generated as described herein may be introduced into a biological system (e.g., a virus, prokaryotic or eukaryotic cell, zygote, embryo, plant, or animal, e.g., non-human animal). A prokaryotic cell may be a bacterial cell. A eukaryotic cell may be, e.g., a fungal (e.g., yeast), invertebrate (e.g., insect, worm), plant, vertebrate (e.g., mammalian, avian) cell. A mammalian cell may be, e.g., a mouse, rat, non-human primate, or human cell. A cell may be of any type, tissue layer, tissue, or organ of origin. In some embodiments a cell may be, e.g., an immune system cell such as a lymphocyte or macrophage, a fibroblast, a muscle cell, a fat cell, an epithelial cell, or an endothelial cell. A cell may be a member of a cell line, which may be an immortalized mammalian cell line capable of proliferating indefinitely in culture.

In some embodiments a nucleic acid that has been modified or generated as described herein may be introduced into a biological system and used to produce a polypeptide or RNA of interest. For example, the nucleic acid may be an expression vector, in which one or more expression control elements, e.g., a promoter, are operably linked, to a sequence that encodes an RNA or protein of interest. The expression vector may be introduced into a cell, which is maintained in culture and produces the polypeptide or RNA of interest. The polypeptide or RNA of interest may be isolated from the cell or may be secreted by the cell and isolated from culture medium. In some embodiments a nucleic acid modified or generated as described herein may be used to generate a transgenic animal or plant.

In some aspects, the invention provides kits useful for performing one or more of the methods of modifying a target nucleic acid. In some embodiments, a kit comprises a Cas enzyme, an exonuclease, a polymerase, and a ligase. In some embodiments a kit comprises one or more containers containing one or more of the enzymes. In some embodiments a kit comprises a container comprising a composition comprising at least two, three, or all four of the enzymes. In some embodiments one or more of the other enzyme(s) may be provided in one or more separate containers. For example, in some embodiments a kit comprises a first container containing a Cas protein and a second container containing an exonuclease, a polymerase, and a ligase. In some embodiments the 4 enzymes may be provided in a mixture in amounts optimized for efficient cloning according to methods described herein. In some embodiments a kit may contain nucleotides (e.g., dNTPs), a buffer, a salt (e.g., MgCl₂) for use in a reaction mixture in which to perform a method described herein. Such components may be provided as a mixture together with one or more of the enzymes or in a separate container. In some embodiments a kit may comprise one more additional components useful in certain methods, such as competent cells (e.g., E. coli), a culture medium for the cells, a positive control for testing a method performed using the kit. In some embodiments a kit may comprise instructions for performing a method described herein.

The foregoing written specification is considered to be sufficient to enable one skilled in the art to practice the invention. Various modifications of the invention in addition to those shown and described herein will become apparent to those skilled in the art from the foregoing description and fall within the scope of the appended claims. The advantages and objects of the invention are not necessarily encompassed by each embodiment of the invention. Those skilled in the art will recognize, or be able to ascertain using no more than routine experimentation, many equivalents to the specific embodiments described herein, which fall within the scope of the claims. The scope of the present invention is not to be limited by or to embodiments or examples described above.

Section headings used herein are not to be construed as limiting in any way. It is expressly contemplated that subject matter presented under any section heading may be applicable to any aspect or embodiment described herein.

Embodiments or aspects herein may be directed to any agent, composition, article, kit, and/or method described herein. It is contemplated that any one or more embodiments or aspects can be freely combined with any one or more other embodiments or aspects whenever appropriate. For example, any combination of two or more agents, compositions, articles, kits, and/or methods that are not mutually inconsistent, is provided.

Articles such as “a”, “an”, “the” and the like, may mean one or more than one unless indicated to the contrary or otherwise evident from the context.

The phrase “and/or” as used herein in the specification and in the claims, should be understood to mean “either or both” of the elements so conjoined. Multiple elements listed with “and/or” should be construed in the same fashion, i.e., “one or more” of the elements so conjoined. Other elements may optionally be present other than the elements specifically identified by the “and/or” clause. As used herein in the specification and in the claims, “or” should be understood to have the same meaning as “and/or” as defined above. For example, when used in a list of elements, “or” or “and/or” shall be interpreted as being inclusive, i.e., the inclusion of at least one, but optionally more than one, of list of elements, and, optionally, additional unlisted elements. Only terms clearly indicative to the contrary, such as “only one of” or “exactly one of” will refer to the inclusion of exactly one element of a number or list of elements. Thus claims that include “or” between one or more members of a group are considered satisfied if one, more than one, or all of the group members are present, employed in, or otherwise relevant to a given product or process unless indicated to the contrary. Embodiments are provided in which exactly one member of the group is present, employed in, or otherwise relevant to a given product or process. Embodiments are provided in which more than one, or all of the group members are present, employed in, or otherwise relevant to a given product or process. Any one or more claims may be amended to explicitly exclude any embodiment, aspect, feature, element, or characteristic, or any combination thereof. Any one or more claims may be amended to exclude any agent, composition, target nucleic acid, or combination thereof.

Embodiments in which any one or more limitations, elements, clauses, descriptive terms, etc., of any claim (or relevant description from elsewhere in the specification) is introduced into another claim are provided. For example, a claim that is dependent on another claim may be modified to include one or more elements or limitations found in any other claim that is dependent on the same base claim. It is expressly contemplated that any amendment to a genus or generic claim may be applied to any species of the genus or any species claim that incorporates or depends on the generic claim.

Where a claim recites a method, a composition for performing the method is provided. Where elements are presented as lists or groups, each subgroup is also disclosed. It should also be understood that, in general, where embodiments or aspects is/are referred to herein as comprising particular element(s), feature(s), agent(s), substance(s), step(s), etc., (or combinations thereof), certain embodiments or aspects may consist of, or consist essentially of, such element(s), feature(s), agent(s), substance(s), step(s), etc. (or combinations thereof). It should also be understood that, unless clearly indicated to the contrary, in any methods claimed herein that include more than one step or act, the order of the steps or acts of the method is not necessarily limited to the order in which the steps or acts of the method are recited.

Where ranges are given herein, embodiments in which the endpoints are included, embodiments in which both endpoints are excluded, and embodiments in which one endpoint is included and the other is excluded, are provided. It should be assumed that both endpoints are included unless indicated otherwise. Unless otherwise indicated or otherwise evident from the context and understanding of one of ordinary skill in the art, values that are expressed as ranges can assume any specific value or subrange within the stated ranges in various embodiments, to the tenth of the unit of the lower limit of the range, unless the context clearly dictates otherwise. “About” in reference to a numerical value generally refers to a range of values that fall within ±10%, in some embodiments ±5%, in some embodiments ±1%, in some embodiments ±0.5% of the value unless otherwise stated or otherwise evident from the context. In any embodiment in which a numerical value is prefaced by “about”, an embodiment in which the exact value is recited is provided. Where an embodiment in which a numerical value is not prefaced by “about” is provided, an embodiment in which the value is prefaced by “about” is also provided. Where a range is preceded by “about”, embodiments are provided in which “about” applies to the lower limit and to the upper limit of the range or to either the lower or the upper limit, unless the context clearly dictates otherwise. Where a phrase such as “at least”, “up to”, “no more than”, or similar phrases, precedes a series of numbers, it is to be understood that the phrase applies to each number in the list in various embodiments (it being understood that, depending on the context, 100% of a value, e.g., a value expressed as a percentage, may be an upper limit), unless the context clearly dictates otherwise. For example, “at least 1, 2, or 3” should be understood to mean “at least 1, at least 2, or at least 3” in various embodiments. It will also be understood that any and all reasonable lower limits and upper limits are expressly contemplated.

Exemplification

### EXAMPLE 1

As described herein, the use of highly specific CRISPR targeting methods linearize plasmids in a short (e.g., 1 hour) isothermal reaction, which can be combined with Gibson-style cloning in a one-step reaction for cutting and assembly of multiple DNA fragments. A sequence requirement for CRISPR-based targeting is a unique target sequence (e.g., about 20 nucleotides) specific to the targeted genomic region and a proto-spacer adjacent motif (PAM) immediately following the guide target sequence. The Cas9 variant of CRISPR commonly used for in vivo genome editing requires a short (NGG) PAM. The target nucleic acid sequence is targeted by guide RNA in a highly specific manner. Genome engineering using the CRISPR/Cas system has been described in Ran et. al., Nature Protocols, 8(11):2281-2308 (2013), incorporated herein in its entirety.

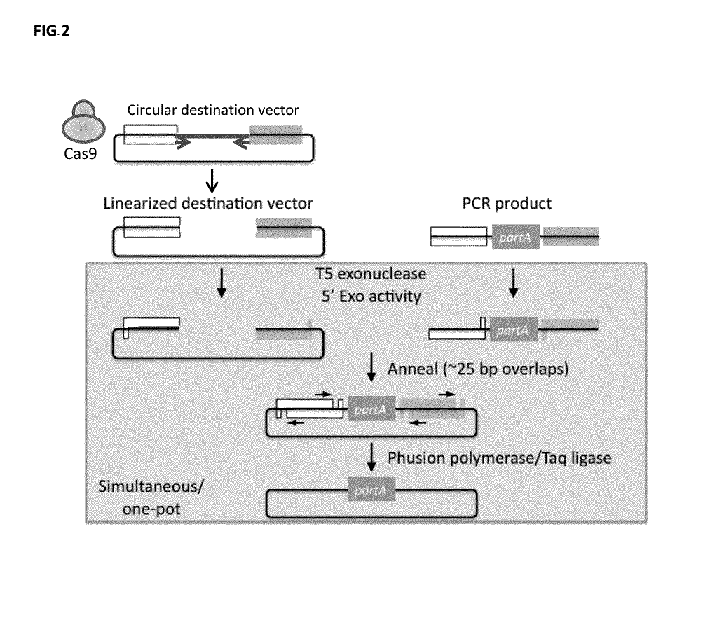

Due to the specificity of the guide RNA, linearizing a plasmid is done with little restrictions and allows excising fragments within genes, promoters, and even sequences overlapping single nucleotide variants (SNVs). In order to assemble new fragments following the plasmid linearization, alternative fragments are designed with overlapping sequences to the desired insertion site (FIG. 2).

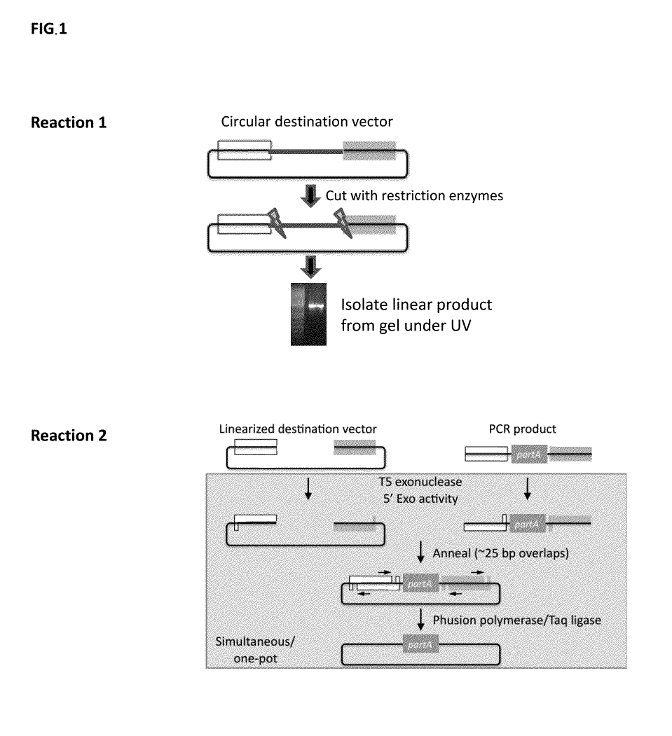

This approach facilitates the use of Gibson assembly to efficiently substitute, delete, insert, or otherwise modify almost any sequence into any destination vector. In addition, the utilization of CRISPR targeting and appropriate guide RNAs can eliminate the need of isolating linear plasmids in reactions (e.g., a Gibson assembly) where sequences are not needed to be replaced. A typical reaction to linearize plasmid, as shown in FIG. 1, includes: incubating the plasmid with restriction enzymes, separating the linear plasmid product by gel electrophoresis, isolating the plasmid by viewing using ultra-violet light, and extracting the plasmid from the agarose gel section.

This process requires an additional reaction and adds considerable hands-on time. With the utilization of CRISPR-based linearization, linearization of a plasmid and assembly (cloning) reaction can take place in the same tube with a single enzyme and guide RNA mix.

### EXAMPLE 2

Using CRISPR Targeting for a Single Reaction Gibson Cloning

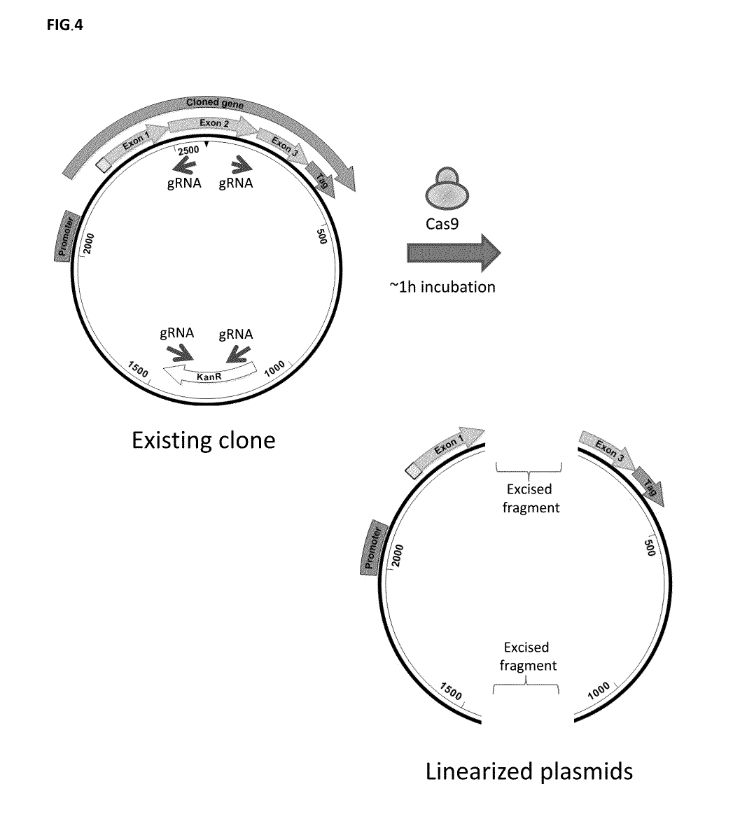

Gibson cloning allows stitching (e.g. assembling) of multiple fragments in a single reaction. Gibson cloning can be difficult in numerous scenarios, for instance, where one part (e.g., a target nucleic acid sequence) of a plasmid to be replaced (e.g., a part of a gene, a plasmid backbone feature, a tag on gene, a promoter, a UTR, etc.) lacks suitable restriction sites or a need to generate many or very large PCR products. Moreover, Gibson cloning works with linearized products (i.e., nucleic acids). See, for example, FIG. 4.

Replacing sequences in plasmids requires unique compatible sequences (see FIG. 1). In order to replace a plasmid segment, it is essential to have unique restriction sites flanking the segment, unique recombination sites (e.g., ATT site, Gateway site, etc.), or the ability to make large PCR products that can be used in a Gibson assembly. These all present a limitation and challenge for many common molecular biology goals.

The approach described herein removes specific segments of plasmid using CRISPR targeting. Guide RNAs (in red, see FIG. 4), are designed against the boundaries of the excised segments of target nucleic acid. CRISPR targeting of any unique sequence (e.g. greater than or about 20 base pairs) allows using any sequence adjacent to a PAM.

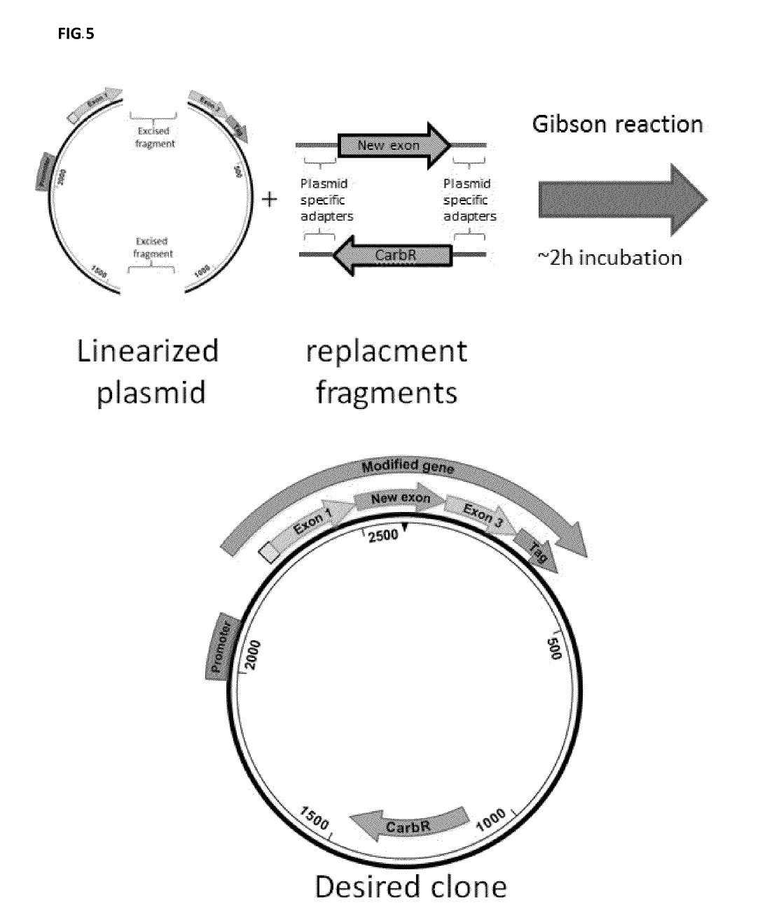

A single reaction modifies (e.g., introduces) the desired fragments to the plasmid. The replacement fragments are introduced with sequences that match the plasmid or their adjacent fragments during a PCR reaction. In one aspect, the replacement fragments have compatible overhangs (e.g., added using, for example, a polymerase chain reaction (PCR), synthetic synthesis of nucleic acids and the like) that match the plasmid or fragment with which they interact. See FIGS. 3, 4 and 5.

As appreciated by one of ordinary skill in the art, there are at least several advantages of using CRISPR targeting for molecular cloning. For most molecular biology applications, these methods can convert any plasmid to have any desired feature, with the existence of the PAM sequence as the major restriction in most scenarios. Also, plasmids do not have to be linearized by other methods. This, thus, eliminates the need to separate linearized plasmid by gel electrophoresis, isolate the plasmid using UV light, and extract the plasmid from the gel. Moreover, the methods described herein, can take place in a single tube, vial, or the like, and the process can be completed in about 2-3 hours. In some embodiments, a single mixture of necessary enzymes and gRNAs can be used for the entire reaction. (see FIG. 2). Linearization of the plasmid and cloning can be performed at or near the same time. In FIG. 2, the vector specific guide RNAs are shown as red arrows.

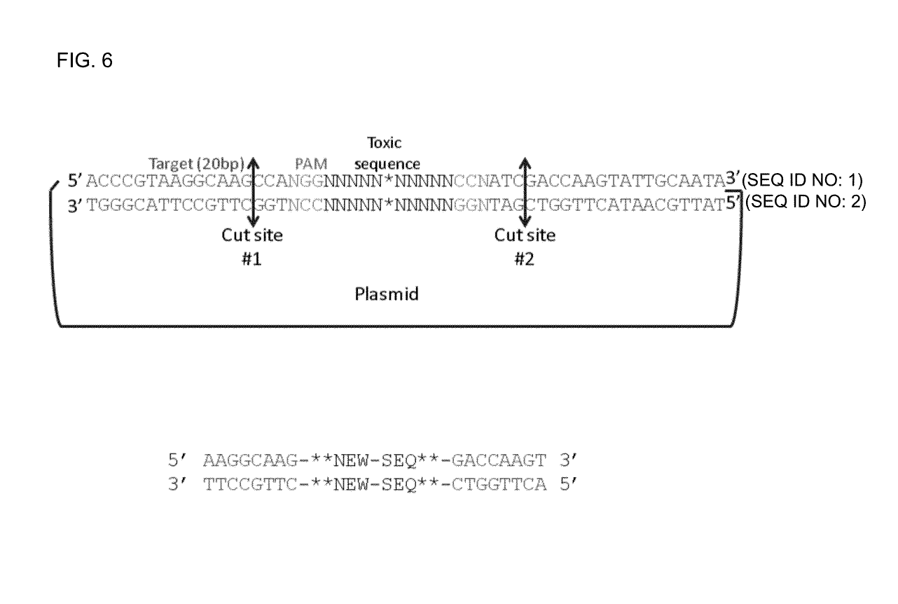

FIG. 6, for example, is one embodiment of the present invention. FIG. 6 shows an exemplary double stranded (ds) DNA sequence on a plasmid. A target sequence of about 20 base pairs and PAM sequence, adjacent to the target sequence, are shown. The toxic sequence or sequence to be modified is also shown. Shown below the plasmid is the fragment to be used for cloning. This sequence includes flanking sequences that overlap with the plasmid. Shown in red, the sequence is part of the target sequence on the plasmid, excluding the PAM and a few bases.

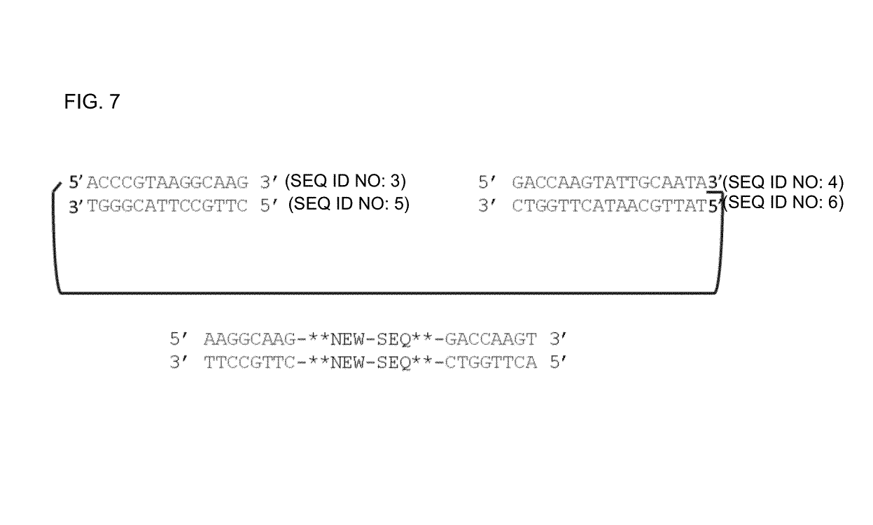

FIG. 7 shows removal of the target nucleic acid sequence within the plasmid using Cas9. Cas9 generates blunt ends, producing a linear plasmid. Moreover, the fragment is not affected by Cas9, since it does not contain a full recognition sequence.

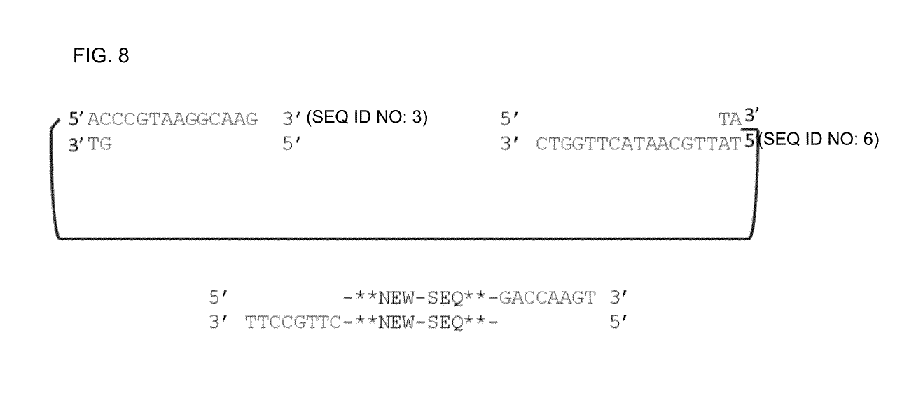

FIG. 8 shows the generation of 3′ overhangs in both the linearized plasmid and fragment (i.e., insert) by an exonuclease.

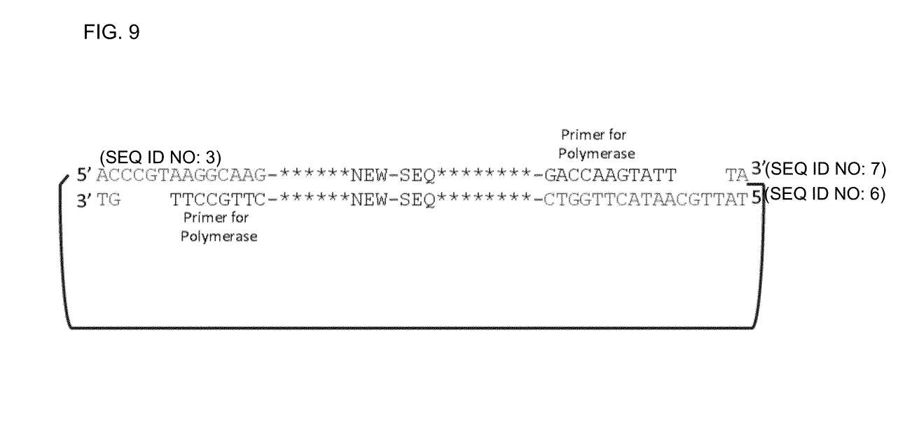

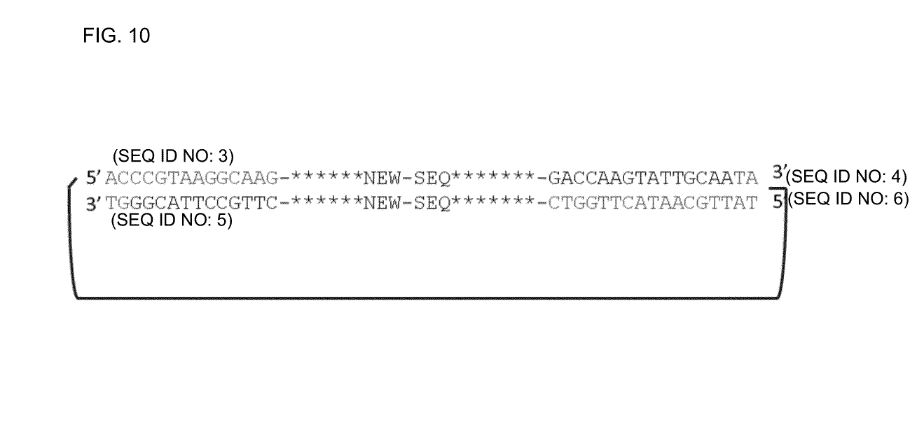

FIG. 9 shows the plasmid and fragment (i.e., insert) complement and prime each other. A DNA polymerase and ligase generate a complete plasmid sequence, shown below. The lack of a PAM and full target sequence prohibit Cas9 to work on the newly completed plasmid. FIG. 10 shows the completed plasmid with insert.

It will be appreciated by one of ordinary skill in the art, that it is possible that Cas9 might cut in only one of the two desired sites. Plasmids with single cuts will not serve as a proper target for cloning, however the presence of the 5′ exonuclease will effectively degrade and remove them from the reaction. It is also an aspect of the invention to devise cloning strategies using a replacement of a negative selection marker in a suitable plasmid. In some aspects, a positive selector fragment can be added.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A method of modifying one or more target nucleic acid sequences comprising:
(a) contacting the one or more target nucleic acid sequences with:
i) one or more ribonucleic acid (RNA) sequences wherein each RNA sequence comprises a portion that is complementary to all or a portion of one or more of the target nucleic acid sequences;
ii) a CRISPR associated (Cas) protein having nuclease activity;
iii) one or more exogenous nucleic acid sequences wherein at least one exogenous nucleic acid sequence comprises a 5′ adapter sequence that hybridizes to a 5′ flanking sequence of the target nucleic acid sequence and at least one exogenous nucleic acid sequence comprises a 3′ adapter sequence that hybridizes to a 3′ flanking sequence of the target nucleic acid sequence; and
iv) a nucleic acid sequence that interacts with Cas protein;
thereby producing a combination; and
(b) maintaining the combination under conditions in which the one or more RNA sequences hybridize to all or the portion of the one or more target nucleic acid sequences to which each RNA sequence forms a complement thereby forming one or more base paired structures, and the one or more base paired structures and the nucleic acid sequence that interacts with Cas protein direct Cas protein to cleave the one or more target nucleic acid sequences;
thereby modifying the one or more target nucleic acid sequences.

2. The method of claim 1, wherein at least one exogenous nucleic acid sequence comprises one or more additional nucleotides.

3. The method of claim 1, wherein at least one exogenous nucleic acid sequences comprises a 5′ adapter sequence and a 3′ adapter sequence.

4. The method of claim 3, wherein the exogenous nucleic acid sequence further comprises one or more additional nucleotides between the 5′ adapter sequence and the 3′ adapter sequence.

5. The method of claim 1, further comprising contacting the combination with one or more exonucleases, polymerases and ligases.

6. (canceled)

7. (canceled)

8. The method of claim 2, wherein the one or more additional nucleotides is a gene, a regulatory sequence, a nucleotide variant, a restriction site, a cloning site, a recombination site, a RNA sequence, portions thereof, or combinations thereof.

9-12. (canceled)

13. The method of claim 2, wherein the exogenous nucleic acid sequence further comprises an additional nucleic acid sequence that encodes a polypeptide.

14. The method of claim 13, wherein the polypeptide is all or a portion of a tag, a transcription factor, an enzyme, a cytokine, a receptor, a transporter, a secreted protein, a binding protein, a post-translational modifying protein, a post-transcriptional modifying protein, a cytoskeletal protein, portions thereof, or combinations thereof.

15-17. (canceled)

18. The method of claim 1, wherein the one or more target nucleic acid sequences comprises a plasmid, a plastid, a bacterial nucleic acid, a bacterial artificial chromosome, a viral nucleic acid, a mitochondrial nucleic acid, or an artificially synthesized nucleic acid.

19. (canceled)

20. The method of claim 1, wherein the Cas protein is Cas9.

21. (canceled)

22. The method of claim 1, wherein the RNA sequence and the nucleic acid sequence that interacts with Cas protein are included in the same sequence.

23. (canceled)

24. (canceled)

25. A method of introducing one or more exogenous nucleic acid sequences into one or more circular nucleic acid sequences comprising:
(a) contacting the one or more circular nucleic acid sequences with:
i) one or more ribonucleic acid (RNA) sequences wherein each RNA sequence comprises a portion that is complementary to all or a portion of one or more target sequences within the one or more circular nucleic acid sequences;
ii) a CRISPR associated (Cas) protein having nuclease activity;
iii) one or more exogenous nucleic acid sequences wherein at least one exogenous nucleic acid sequence comprises a 5′ adapter sequence that hybridizes to a 5′ flanking sequence of the target nucleic acid sequence and at least one exogenous nucleic acid sequence comprises a 3′ adapter sequence that hybridizes to a 3′ flanking sequence of the target nucleic acid sequence; wherein at least one exogenous nucleic acid sequence comprises one or more additional nucleotides; and
iv) a nucleic acid sequence that interacts with Cas protein;
thereby producing a combination; and
(b) maintaining the combination under conditions in which the one or more RNA sequences hybridize to all or the portion of the one or more target nucleic acid sequences to which each RNA sequence forms a complement thereby forming one or more base paired structures, and the one or more base paired structures and the nucleic acid sequence that interacts with Cas protein direct the Cas protein to cleave the target nucleic acid sequence;
thereby introducing the one or more exogenous nucleic acid sequence into the one or more circular nucleic acid sequences.

26. The method of claim 25, wherein at least one exogenous nucleic acid sequence comprises a 5′ adapter sequence and a 3′ adapter sequence.

27. The method of claim 26, wherein the exogenous nucleic acid sequence comprises the one or more additional nucleotides between the 5′ adapter sequence and the 3′ adapter sequence.

28. The method of claim 27, further comprising contacting the combination with one or more exonucleases, polymerases and ligases.

29. (canceled)

30. (canceled)

31. The method of claim 27, wherein the one or more additional nucleotides comprises a gene or portion thereof, a regulatory sequence, a nucleotide variant, a restriction site, a cloning site, a recombination site, a RNA sequence, portions thereof, or combinations thereof.

32-35. (canceled)

36. The method of claim 27, wherein the additional nucleotide comprises a nucleic acid sequence that encodes a polypeptide.

37-40. (canceled)

41. The method of claim 25, wherein the one or more circular nucleic acid sequences comprise a plasmid, a plastid, a bacterial nucleic acid, a bacterial artificial chromosome, a viral nucleic acid, a mitochondrial nucleic acid, or an artificially synthesized nucleic acid.

42. The method of claim 25, wherein the Cas protein is Cas9.

43. (canceled)

44. The method of claim 25, wherein the RNA sequence and the nucleic acid that interacts with Cas protein are on the same sequence.

45. (canceled)

46. (canceled)
